(19) United States
(12) Patent Application Publication
(10) Pub. No.: US 2025/0236828 A1
(43) Pub. Date: Jul. 24, 2025

(54) CELL CULTURE PLATFORM

(71) Applicants: I-Shou University, Kaohsiung City (TW); E-DA Hospital, Kaohsiung City (TW)

(72) Inventors: Li-Feng Liu, Kaohsiung City (TW); Li-Yen Shiu, Kaohsiung City (TW)

(21) Appl. No.: 18/745,154

(22) Filed: Jun. 17, 2024

(30) Foreign Application Priority Data
Jan. 19, 2024 (TW) 113102330

(51) Int. Cl. C12M 1/32; C12M 1/12

(52) U.S. Cl. CPC C12M 23/12; C12M 25/06

(57) ABSTRACT

A cell culture platform includes a base plate, multiple through holes formed in the base plate, and a support wall extending downward from the periphery of the base plate. The base plate has a top surface and a bottom surface opposite to the top surface. The through holes traverse the top surface and bottom surface of the base plate. Each of the through holes has an upper opening and a lower opening, and the aperture gradually tapers from the upper opening to the lower opening. The support wall defines an accommodating space beneath the base plate. The structural design of the cell culture platform allows for the formation of larger hanging drop during cell suspension culture. Additionally, the cell culture platform can be used as an adapter, which is compatible with covers and 96-well plates of any brand.

### RELATED APPLICATIONS

This application claims priority of Taiwan Application No. 113102330, filed on Jan. 19, 2024. The entire disclosures of all the above applications are hereby incorporated by reference.

### FIELD OF THE INVENTION

The present invention relates to a cell culture platform, particularly a cell culture platform that provides a better space for growth.

### BACKGROUND OF THE INVENTION

2D cell culture is a traditional cell culture method, which refers to suspending cells on a flat culture dish (such as a cell culture plate or culture dish) so that the cells attach to the flat surface and grow. It has the advantages of easy control and operation. In 2D cell culture, the cell morphology usually presents a flat shape, which is not conducive to simulating the true shape of cells in vivo. At the same time, it is also unable to simulate the growth status of cells in the 3D environment of the body, which may lead to significant differences in cell behavior from that in the body.

Further, when the cells are moved from the culture dish to other containers for various tests, some cells may be lost during the movement, resulting in a decrease in cell number. In addition, if the movement process is not performed carefully, it may also affect cell activity.

### SUMMARY OF THE INVENTION

One object of the present invention is to provide a cell culture platform that promotes cultured cells to be closer to in vivo growth conditions and facilitates the transfer of cell clusters.

The cell culture platform comprises a base plate, a plurality of through holes formed on the base plate, and a support wall extends from the base plate. The base plate comprises a top surface and a bottom surface opposite to the top surface. Each of the through holes penetrates the top surface and the bottom surface and comprises an upper opening and a lower opening. The aperture of each said through holes is tapered from the upper opening to the lower opening. The support wall extends downward from the periphery of the base plate and surrounds an accommodating space.

In a preferable embodiment, the base plate further comprises a plurality of support rings protruding downward from the bottom surface, the support rings are in the accommodating space and are arranged corresponding to the through holes, and each said support rings surrounds the lower opening corresponding to each said through holes.

In a preferable embodiment, the lower opening of each said through holes protrudes from the bottom surface of the base plate and is in the accommodating space.

In a preferable embodiment, there is a first distance between the top surface and the bottom surface of the base plate, there is a second distance between the upper opening and the lower opening of each said through holes, and the second distance is greater than the first distance.

In a preferable embodiment, the diameter of the upper opening of each said through holes is greater than 4 mm, and the second distance is greater than 10 mm.

In a preferable embodiment, the diameter of the upper opening of each said through holes is 5 mm, and the second distance is 12 mm.

In a preferable embodiment, each said through holes is formed by a surrounding wall, the surrounding wall has an inclination angle, and the inclination angle is between 4° and 6°, including the endpoint values.

In a preferable embodiment, the maximum width of the base plate is smaller than the maximum width of the support wall, and the support wall comprises at least one abutment portion located in the accommodating space.

In a preferable embodiment, the base plate further comprises four alignment portions located at the corners.

In a preferable embodiment, the through holes are arranged in a matrix.

The characteristic of the present invention is that the structure of the through holes of the cell culture platform can form larger hanging drops when cells are cultured in hanging drops, allowing cells to grow into larger 3D cell clusters, which can better simulate the growth conditions of cells in vivo. Furthermore, this cell culture platform can be used as an adapter and used with any brand of cover and 96-well plate.

### BRIEF DESCRIPTION OF THE DRAWINGS

FIG. 1 is a perspective view of a preferred embodiment of the cell culture platform of the present invention.

FIG. 2 is a top view to assist in explaining FIG. 1.

FIG. 3 is a cross-sectional side view illustrating the structure along the section line III-III in FIG. 2.

FIG. 4 is a schematic diagram illustrating an enlargement of the frame A in FIG. 3.

FIG. 5 is an exploded perspective view illustrating that the preferred embodiment is used in conjunction with an upper cover and a 96-well plate.

FIG. 6 is a cross-sectional side view illustrating the cross-sectional structure of the cell culture platform combined with the 96-well plate.

FIG. 7 is a schematic diagram illustrating an enlargement of the frame B in FIG. 6.

FIG. 8 is a statistical chart comparing the size of cell spheroids of fibroblasts cultured in this preferred embodiment and in a low-adhesion 96-well plate.

FIG. 9 is a statistical chart comparing the size of cell spheroids of colorectal tumor cells cultured in this preferred embodiment and in a low-adhesion 96-well plate.

FIG. 10 is a schematic diagram illustrating the morphology of fibroblasts cultured with different concentrations of gelatin for seven days.

FIG. 11 is a schematic diagram illustrating the morphology of colorectal tumor cells cultured with different concentrations of gelatin for seven days.

### DETAILED DESCRIPTION OF THE INVENTION

The detailed description and preferred embodiments of the invention will be set forth in the following content and provided for people skilled in the art to understand the characteristics of the invention. Directional terms mentioned in the following embodiments, such as up, down, left, right, front, back, bottom, top, etc., are only for reference to the directions in the attached drawings. Therefore, the directional terms used are for explanation and not limitation of the invention.

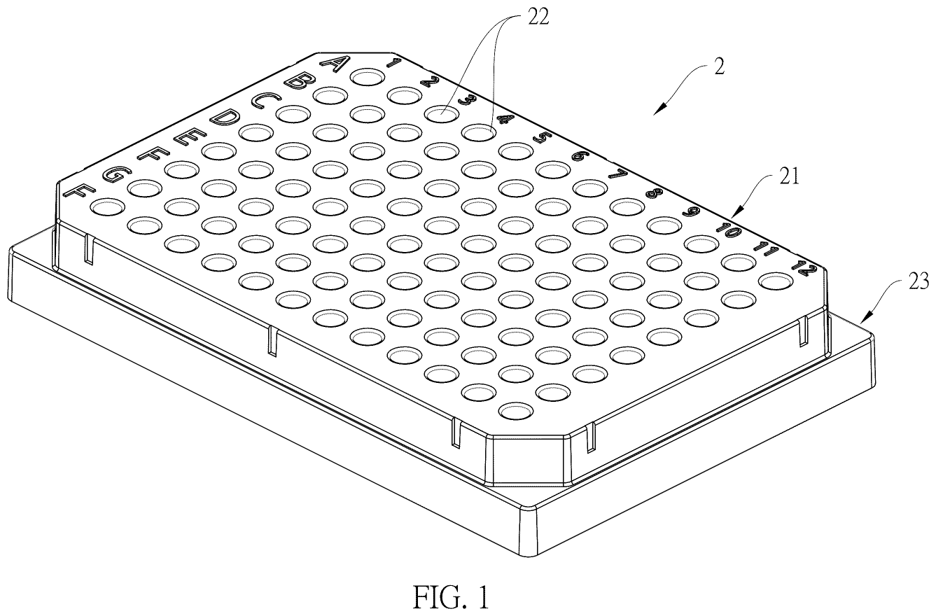

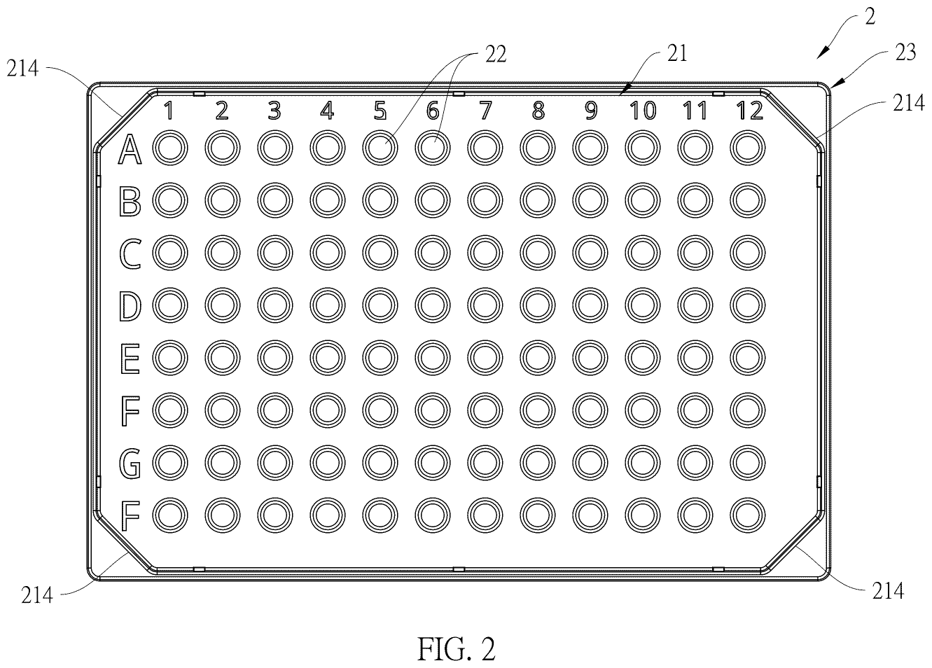

Referring to FIG. 1 and FIG. 2, it is a preferred embodiment of a cell culture platform 2 of the present invention. The cell culture platform 2 comprises a base plate 21, a plurality of through holes 22 formed in the base plate 21, and a support wall 23 extending downward from the periphery of the base plate 21.

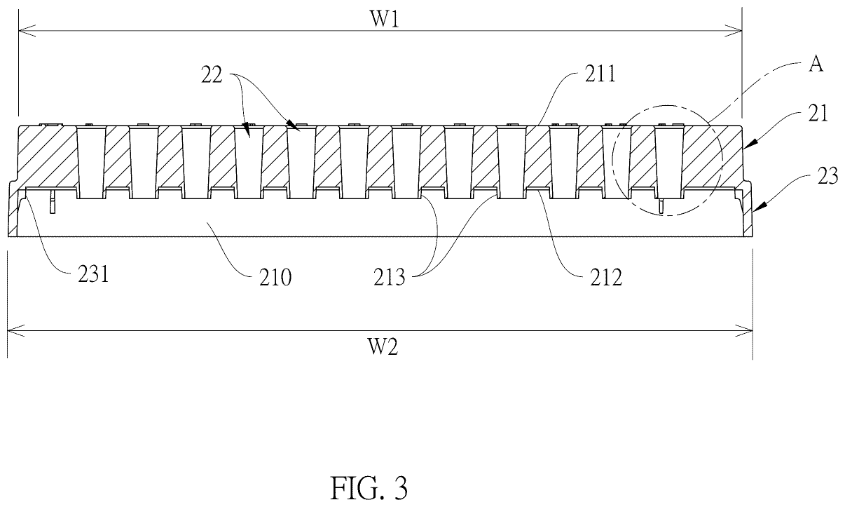

Referring to FIG. 3, the base plate 21 comprises a top surface 211, a bottom surface 212 opposite to the top surface 211, and a plurality of support rings 213 protruding downward from the bottom surface 212. Each of the through holes 22 penetrates the top surface 211 and the bottom surface 212 of the base plate 21, and the support rings 213 are provided corresponding to the through holes 22. The support wall 23 surrounds an accommodating space 210 below the base plate 21, and the support rings 213 are in the accommodating space 210. Wherein, the maximum width W1 of the base plate 21 is smaller than the maximum width W2 of the support wall 23. The support wall 23 comprises at least one abutment portion 231 located in the accommodating space 210. The abutment portion 231 is located at the inner side of the support wall 23.

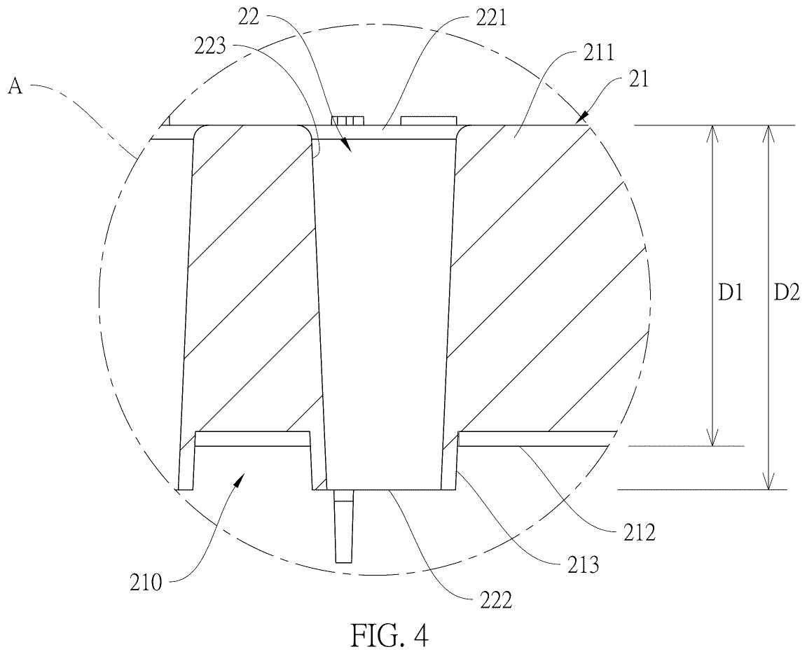

Referring to FIG. 4, which is an enlargement of the frame A in FIG. 3, each of the through holes 22 has an upper opening 221 and a lower opening 222. Each said support ring 213 surrounds the lower opening 222 of each corresponding through holes 22. In more detail, since the support rings 213 are in the accommodating space 210, the lower opening 222 of each said through holes 22 also protrudes from the bottom surface 212 of the base plate 21 and is in the accommodating space 210. There is a first distance D1 between the top surface 211 and the bottom surface 212 of the base plate 21, there is a second distance D2 between the upper opening 221 and the lower opening 222 of each said through holes 22, and the second distance D2 is greater than the first distance D1. In addition, the diameter of each said through holes 22 is tapered from the upper opening 221 to the lower opening 222. Wherein, each of the through holes 22 is surrounded by a surrounding wall 223. The surrounding wall 223 has an inclination angle, and the inclination angle is between 4° and 6°, including the endpoint values. In this embodiment, the inclination angle is 5°. The diameter of the upper opening 221 of each of the through holes 22 is greater than 4 mm, and the distance between the upper opening 221 and the lower opening 222 of each of the through holes 22 (the second distance D2) is greater than 10 mm. In this embodiment, the diameter of the upper opening 221 of each said through holes 22 is 5 mm, and the second distance D2 is 12.5 mm.

The cell culture platform 2 of the present invention is used for hanging drop culture of cells, which is one of the methods of scaffold-free cell spheroid culture. Cell spheroid culture is a simple 3D culture method that can be used for most cell types. It mainly uses the natural tendency of adherent cells to aggregate and form spheroids when they are hindered from adhering to the culture dish, so that the cells form spheroids. The cell clusters forming spheroids can effectively diffuse oxygen and nutrients and remove metabolic waste, and can be imaged by optical, fluorescence and confocal microscopy to easily analyze the spheroids, which is an advantage of the 3D cell culture model. Therefore, spheroid culture can more effectively simulate avascular tumor behavior and the differentiation of pluripotent stem cells (PSCs) than 2D environment.

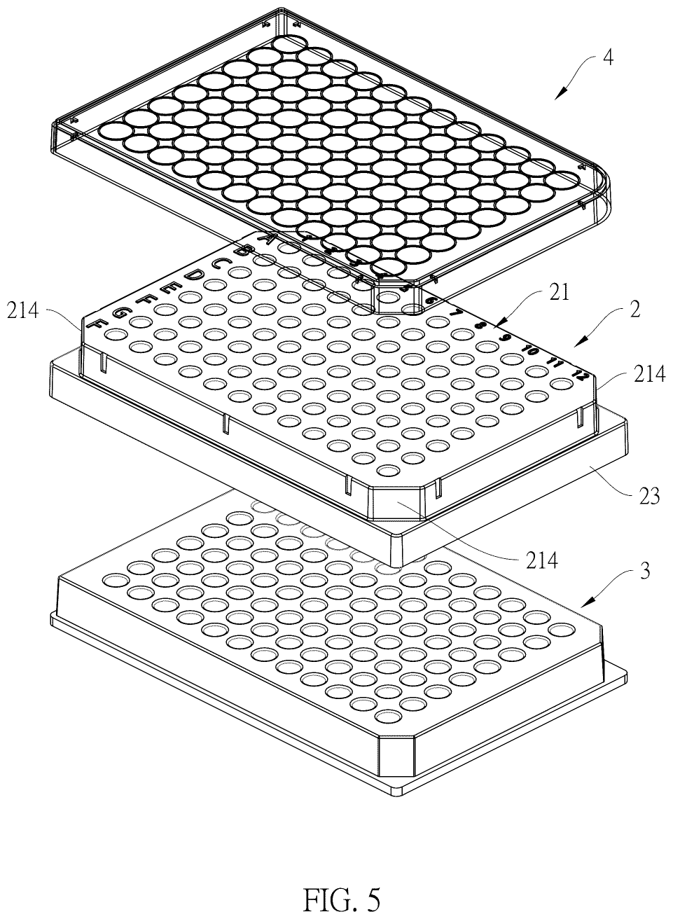

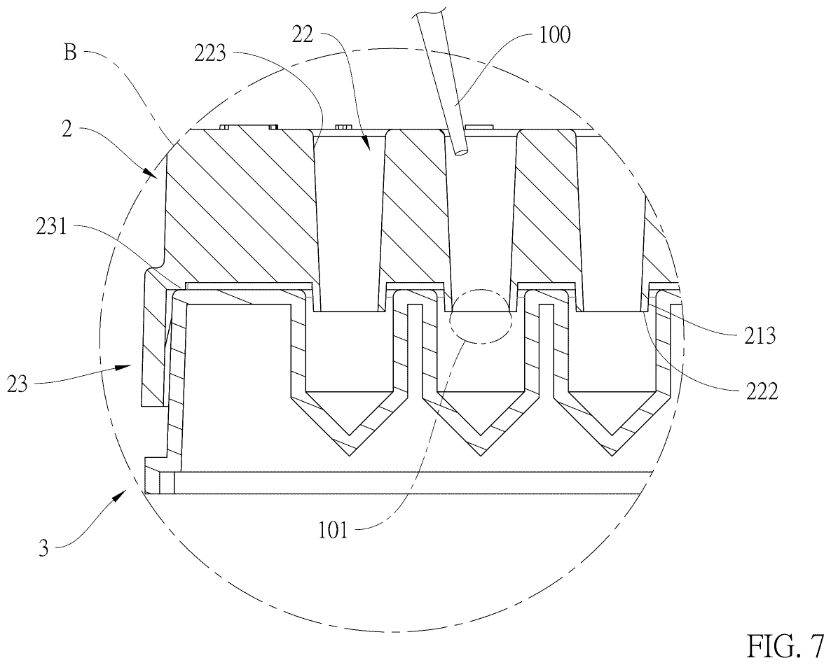

The most common type of conventional cell culture equipment is a 96-well plate with a cover. The wells of the 96-well plate are used to accommodate cells and culture medium, and the cover prevents foreign matter from falling into the 96-well plate. The most important thing is that the cell culture platform 2 of the present invention can be used as an adaptor with any brand and any form of 96-well plate and cover. Therefore, the through holes 22 of the cell culture platform 2 are arranged in a matrix like the 96-well plate. Referring to FIG. 2 and FIG. 5, the cell culture platform 2 of the present invention is used in conjunction with a 96-well plate 3 and a cover 4. The cover 4 is disposed on the base plate 21 of the cell culture platform 2 to cover the through holes 22. The cell culture platform 2 covers the 96-well plate 3 and the support wall 23 is used to abut against the periphery of the 96-well plate 3. In generally, the cover 4 is made of transparent material, and in response to experimental requirements, the cover 4 sometimes has markings corresponding to different wells of the 96-well plate 3. Therefore, a cutting portion is usually formed at one corner of the cover 4 as an alignment indicator when the cover 4 is disposed on the 96-well plate 3. Since the position of the cutting portion of the cover 4 of different brands may be different, the cell culture platform 2 of the present invention is formed with cutting-shaped alignment portions 214 at the four corners of the base plate 21, so the cell culture platform 2 can be used in conjunction with any brand of the cover 4. Taking FIG. 5 as an example, the lower right corner of the cover 4 is a cut shape, and the upper right corner is arc shaped. The four corners of the base plate 21 of the cell culture platform 2 of the present invention are formed with alignment portions 214, so the cover 4 can smoothly cover on the base plate 21. Similarly, in order to align with the cover 4, the corners of the 96-well plate 3 will also be cut in corresponding to the shape of the cover 4. Therefore, in order to be used with any brand of 96-well plate 3, the four corners of the cell culture platform 2 of the present invention will not be cut and can cover on various types of 96-well plate 3. Through the above structural design, the covers 4 of different brands can be disposed on the base plate 21 of the cell culture platform 2, and the cell culture platform 2 can be disposed on various types of 96-well plate 3. It should be noted that the cover 4, the 96-well plate 3, and the cell culture platform 2 can all be transparent, but for the sake of simplicity the lines of the drawing, only the cover 4 is in a transparent state in FIG. 5. At the same time, as shown in FIG. 7, the abutment portion 231 located at the inner side the support wall 23 will abut against the top surface of the 96-well plate 3. The support wall 23 and the abutment portion 231 enable the cell culture platform 2 to cover the 96-well plate 3 stably.

In this embodiment, experiments were conducted using two cell lines: non-cancer cell fibroblasts (NIH-3T3) and colorectal tumor cells (HCT-116). The above-mentioned cell lines are cultured in appropriate culture medium, and gelatin is added to the culture medium to assist the cells to aggregate with each other during the culture process. Since the culture method is not the focus of the present invention and is common knowledge in the art, the details will not be described.

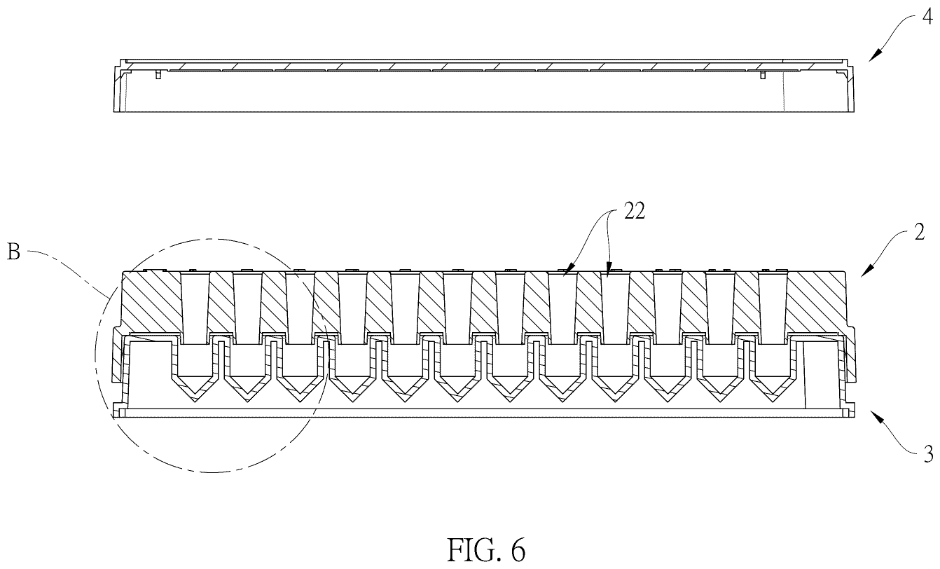

Referring to FIG. 6 in conjunction with FIG. 3, during the cell culture process, the cell culture platform 2 is covered on the 96-well plate 3, and the cover 4 is also covered on the cell culture platform 2. In more detail, the support wall 23 of the cell culture platform 2 is engaged with the outer periphery of the 96-well plate 3, so that the 96-well plate 3 is in the accommodating space 210 surrounded by the support wall 23.

Referring to FIG. 7 in conjunction with FIG. 4, a quantitative pipette 100 is used to slowly inject the culture medium mixed with the above cells into the through holes 22 of the cell culture platform 2. Since the surrounding wall 223 of each of the through holes 22 has an inclination, when the culture medium enters each of the through holes 22, it can slide down smoothly to avoid remaining on the surrounding wall 223, and the hanging drop 101 is formed at the lower opening 222 of each said through holes 22. When the inclination is too large, it will be difficult for the culture medium to flow downward and will remain on the surrounding wall 223. If the inclination is too small (for example, the surrounding wall 223 is completely vertical), the downward flow rate of the culture medium may be too fast, which is not conducive to the formation or maintenance of the hanging drop 101. Since the support ring 213 is formed corresponding to the lower opening 222 of each of the through holes 22, better suspension force can be provided for the hanging drop 101. Therefore, in this embodiment, the surrounding wall 223 of the through hole 22 is designed with a specific inclination, so that the volume of the culture medium added to each of the through holes 22 can be up to 180 μl while still maintaining a hanging drop shape. During the culture process, a quantitative pipette 100 can be used to gently suck up a portion of the culture medium through each of the through holes 22, and then gently inject new culture medium to replace the culture medium. If the volume of culture medium containing cells added initially is 70-80 μl, 20-40 μl of culture medium can be replaced each time. As shown in FIG. 7, since the cell culture platform 2 is disposed on the 96-well plate 3, the hanging drops 101 below each of the through holes 22 will be correspondingly located above the wells of the 96-well plate 3. When the cells are ready to be tested, as long as enough culture medium is added in each of the through holes 22, the hanging drop 101 can directly fall downward into the corresponding well of the 96-well plate 3. Therefore, the 96-well plate 3 can be directedly used for detection or image shooting.

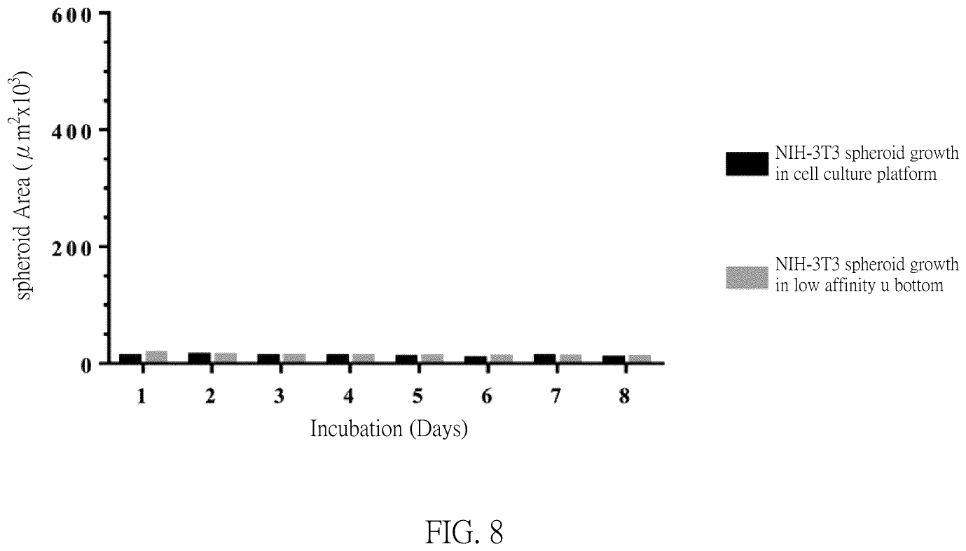

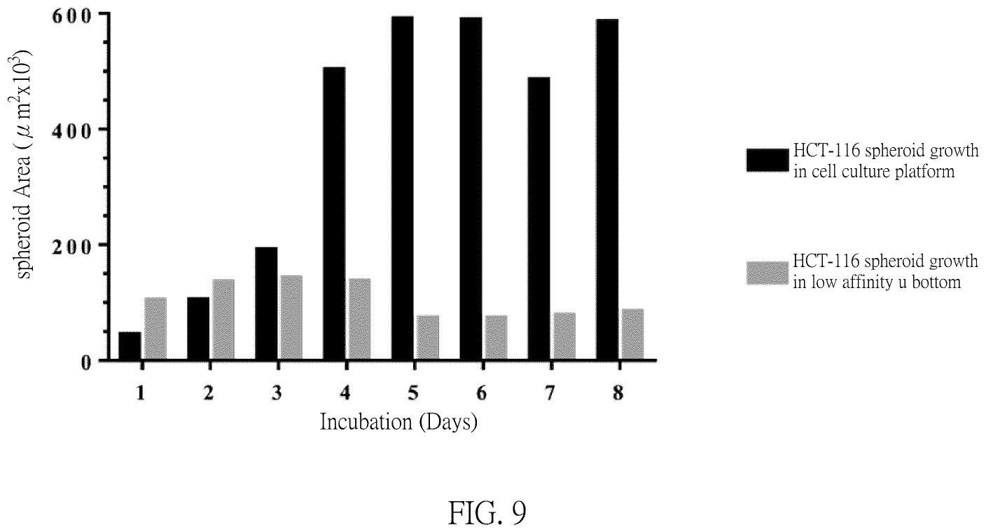

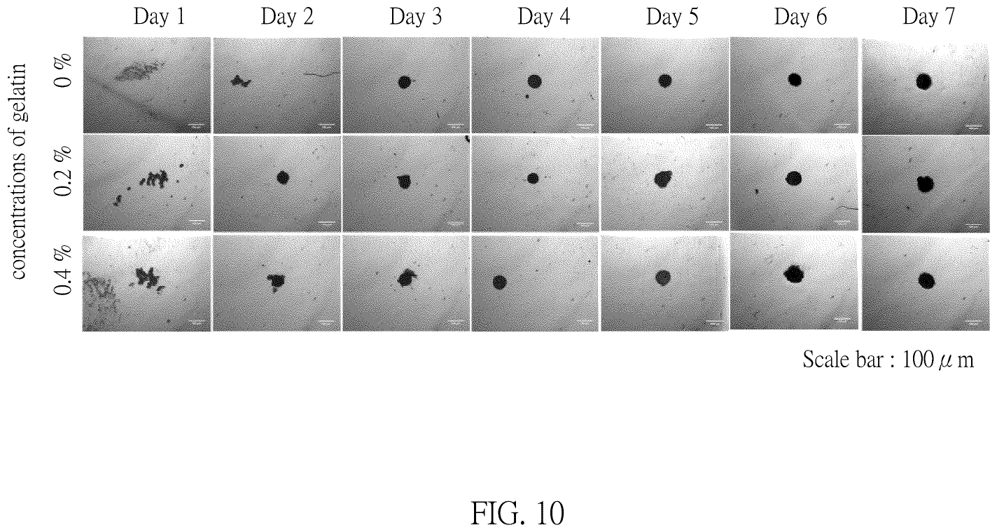

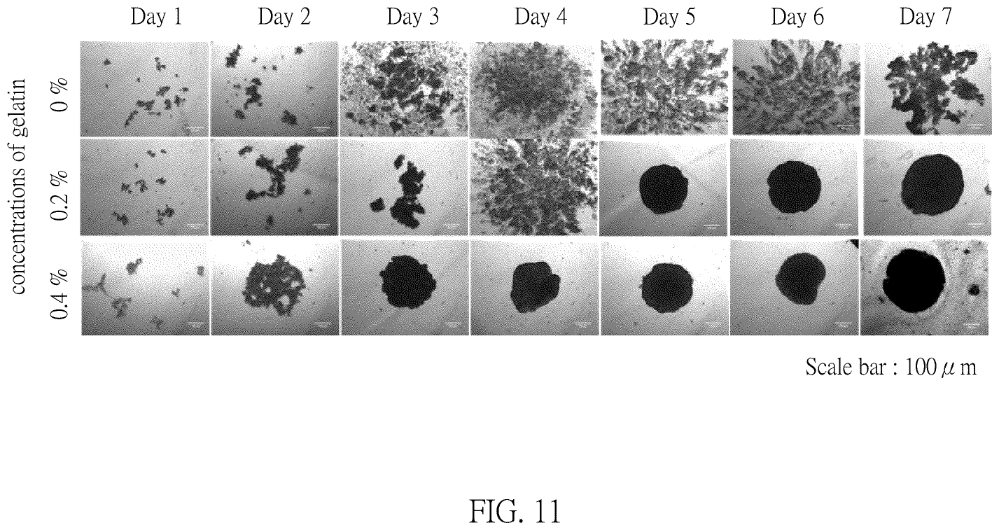

In the experimental results shown in FIG. 8 and FIG. 9, non-cancerous fibroblasts (NIH-3T3) and colorectal tumor cells (HCT-116) were respectively cultured in the cell culture platform 2 of the present invention and in the low affinity 96-well plates. Wherein, the cell culture platform 2 of the present invention is used for the experimental group, and the low affinity culture plate is used for the control group. Low affinity culture plate is a multi-well plate commonly used for 3D cell culture. The bottom of the well is often U-shaped or V-shaped, and the wall of the wells are coated with a low-adhesion coating, which prevents cells from adhering to the wall during culture and instead forms spheres in the middle of the well. FIG. 8 shows the experimental results of fibroblast cells, and FIG. 9 shows the experimental results of colorectal tumor cells. Since fibroblasts are not rapidly proliferating cells and their growth requires adhesion, therefore, referring to FIG. 8, it can be seen from the experimental result that whether the cell culture platform 2 of the present invention or the low affinity culture plate is used for culture, there is no significant difference in the volume of cell spheroids. Referring to FIG. 9, colorectal tumor cells are rapidly proliferating cells and can form spherical structures. Therefore, when the colorectal tumor cells are cultured in the cell culture platform 2 of the present invention, the volume of the cell spheroids will be larger than the volume of the cell spheroids cultured with low affinity culture plate. FIG. 10 and FIG. 11 show that different concentrations of gelatin were added to the culture medium for culture. FIG. 10 shows the culture results of fibroblasts (NIH-3T3), and FIG. 11 shows the culture results of colorectal tumor cells (HCT-116). It can be seen from the culture results that co-culture with 4% gelatin can obviously assist colorectal tumor cells to aggregate into spheres from the third day on. In FIG. 10 and FIG. 11, the scale bar in the photo is 100 μm.

In the cell culture platform 2 of the present invention, the aperture of each said through holes 22 is tapered from the upper opening 221 to the lower opening 222. That is, each said through holes 22 is formed with an inclination so that the culture fluid is less likely to remain on the surrounding wall 223 of the through hole 22. At the same time, the diameter of the upper opening 221 on each of the through holes 22 is greater than 4 mm, and the distance between the upper opening 221 and the lower opening 222 of each said through holes 22 is greater than 10 mm. Therefore, the through holes 22 provide enough space for the culture medium, and the culture medium volume in each of the through holes 22 can be up to 180 μl and can still maintain a hanging drop shape below the through holes 22. In this way, the cells in the hanging drop can be provided with enough growth space to form cell spheroids, and it is also easier to replace the culture medium and extend the culture time, making the cell spheroids larger and be better to simulate the actual state and size of tumor cells in the body. In addition, the cell culture platform 2 of the present invention can be used with any brand of cover 4 and 96-well plate 3. Therefore, when the culture is completed and various tests are to be carried out, as long as enough culture medium is added to each of the through holes 22, the hanging droplets can directly fall downward into the corresponding wells of the 96-well plate 3. It is very convenient for transferring cells, and there will be no residual cells caused by incomplete transfer.

The cell culture platform of the present invention can be used to culture cell spheroids. It has the possibility that the cell spheroids can be further cultured into organoids to provide a cell model closer to clinical practice. At the same time, it can be combined with 3D organoid in vitro detection technology and high-speed image analyzer to observe the expression of specific genes to establish the relationship between target drugs and specific genes. It can also be applied to the screening of drug and the selection of clinical patient medications to achieve the purpose of accurate treatment.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A cell culture platform, comprising:
a base plate, comprising a top surface and a bottom surface opposite to the top surface;
a plurality of through holes, formed on the base plate and penetrating the top surface and the bottom surface, wherein each of the through holes comprises an upper opening and a lower opening, and the aperture of each said through holes is tapered from the upper opening to the lower opening; and
a support wall, extending downward from the periphery of the base plate and surrounding an accommodating space.

2. The cell culture platform as claimed in claim 1, wherein the base plate further comprises a plurality of support rings protruding downward from the bottom surface, the support rings are in the accommodating space and are arranged corresponding to the through holes, and each said support rings surrounds the lower opening corresponding to each said through holes.

3. The cell culture platform as claimed in claim 2, wherein the lower opening of each said through holes protrudes from the bottom surface of the base plate and is in the accommodating space.

4. The cell culture platform as claimed in claim 1, wherein there is a first distance between the top surface and the bottom surface of the base plate, there is a second distance between the upper opening and the lower opening of each said through holes, and the second distance is greater than the first distance.

5. The cell culture platform as claimed in claim 4, wherein the diameter of the upper opening of each said through holes is greater than 4 mm, and the second distance is greater than 10 mm.

6. The cell culture platform as claimed in claim 5, wherein the diameter of the upper opening of each said through holes is 5 mm, and the second distance is 12 mm.

7. The cell culture platform as claimed in claim 1, wherein each said through holes is formed by a surrounding wall, the surrounding wall has an inclination angle, and the inclination angle is between 4° and 6°, including the endpoint values.

8. The cell culture platform as claimed in claim 1, wherein the maximum width of the base plate is smaller than the maximum width of the support wall, and the support wall comprises at least one abutment portion located in the accommodating space.

9. The cell culture platform as claimed in claim 1, wherein the base plate further comprises four alignment portions located at the corners.

10. The cell culture platform as claimed in claim 1, wherein the through holes are arranged in a matrix.
